(19) United States
(12) Patent Application Publication
(10) Pub. No.: US 2019/0298589 A1
(43) Pub. Date: Oct. 3, 2019

(54) NAPPY ACCESSORY

(71) Applicant: Diapers2Go Pty Ltd, Brisbane (AU)

(72) Inventors: Thomas Mai, Brisbane (AU); Scott Farnworth, Brisbane (AU)

(21) Appl. No.: 16/354,620

(22) Filed: Mar. 15, 2019

(30) Foreign Application Priority Data
Mar. 28, 2018 (AU) 2018100383

(51) Int. Cl. A61F 13/84; A47D 5/00

(52) U.S. Cl. CPC A61F 13/84; A47D 5/006

(57) ABSTRACT

A nappy accessory including a pouch attached or attachable to a nappy, and a change mat located in the pouch.

### FIELD OF INVENTION

The present invention relates to a nappy accessory. Reference will be made in the specification to the use of the invention with respect to nappies. The patent specification describes this use but it is by way of example only and the invention is not limited to this use.

### BACKGROUND OF THE INVENTION

Nappies are primarily worn by infants and toddlers, and typically require changing multiple times a day.

A problem arises when a parent or carer is traveling with a child and the nappy requires changing.

To effectively change a nappy a parent or carer typically requires wipes in addition to a spare nappy. To address this problem, parents and carers of infants and toddlers typically carry a nappy bag with spare nappies, wipes and other accessories to help them change a soiled nappy.

Nappy bags are typically quite large and include spare nappies, paper towels, wet wipes, barrier cream or powder, a changing mat and rubbish bags. Such nappy bags are typically kept in a car or with a pram or stroller or the like.

It is impractical for parents or carers to always carry a nappy bag and problems can arise when a nappy requires changing and the nappy bag is not within easy reach.

### OBJECT OF THE INVENTION

It is an object of the present invention to overcome or at least alleviate one or more of the above mentioned problems with changing nappies and/or provide the consumer with a useful or commercial choice.

### SUMMARY OF THE INVENTION

In one aspect, the present invention broadly resides in a nappy accessory including
a pouch attached or attachable to a nappy; and
a change mat located in the pouch.

The pouch is preferably openable to enable access to the contents of the pouch. Preferably the pouch is sealed prior to being opened by a user. In one embodiment, the pouch is resealable. In one embodiment, the pouch is resealable using an adhesive strip. In another embodiment, the pouch is resealable using a hook and loop fastener. In a further embodiment, the pouch is resealable using a plastic zipper opening.

In one embodiment, the pouch has a weakened portion which enables a user to open the pouch. The weakened portion is preferably a perforated portion. Preferably the pouch has a perforation to enable a user to open the pouch along the perforation.

In one embodiment, the pouch is attached to the nappy. Preferably the pouch is glued to a nappy. In another embodiment, the pouch is heat welded to the nappy.

In one embodiment, the pouch includes an adhesive portion. Preferably the pouch includes a removable backing to expose the adhesive portion. Preferably the pouch is attachable to a nappy by adhering the adhesive portion to a portion of the nappy.

In one embodiment, the pouch is releasably attachable to a nappy. Preferably, the pouch includes a hook fastener portion. Preferably the hook fastener is adapted to releasably attach the pouch to a nappy. Preferably the hook fastener portion releasably attaches to fibres of the external fabric of the nappy.

In one embodiment, the pouch includes one or more internal pockets. The one or more internal pockets preferably assist in retaining the contents of the pouch in position.

The change mat is preferably a plastic change mat. The change mat is preferably located in the pouch in a folded configuration. Alternatively, the change mat can be located in the pouch in a rolled-up configuration. The change mat preferably includes a drawstring. The drawstring is preferably located adjacent a periphery of the change mat. Preferably a portion of the change mat is folded over to contain the drawstring. The folded over portion preferably includes one or more apertures to access the drawstring. In a preferred embodiment, the folded over portion includes two apertures to access the drawstring. Preferably the drawstring can be pulled to convert the change mat into a bag. In the embodiment with two apertures to access the drawstring, the drawstring can be pulled from both of the apertures to convert the change mat into a bag, and the two loops of drawstring extending from the apertures can be tied together to securely close the bag.

In one embodiment, the change mat is releasably attached to an inside portion of the pouch. Preferably the change mat includes a tab attached or attachable to an inside portion of the pouch. Preferably the tab is tearable to detach the change mat from the pouch.

The nappy accessory preferably includes one or more wipes located in the pouch. In one embodiment, the one or more wipes are one or more paper towels or tissues.

The nappy accessory preferably includes one or more wet wipes located in the pouch.

The nappy accessory preferably includes one or more packets of barrier cream and/or baby powder located in the pouch. Preferably the baby powder is a corn starch based baby powder.

In one embodiment, the nappy accessory preferably includes one or more gloves located in the pouch. Preferably the nappy accessory includes a pair of gloves.

In another aspect, the present invention broadly resides in a nappy accessory including
a plastic change mat having a drawstring located adjacent a periphery of the change mat,
wherein in use pulling the drawstring converts the change mat to a bag.

The nappy accessory preferably further includes a pouch attachable or attached to a nappy, the pouch housing the change mat prior to use.

Preferably the change mat is housed in the pouch in a folded condition.

In a further aspect, the present invention broadly resides in a nappy accessory including
a pouch attached or attachable to a nappy; and
one or more wipes located in the pouch.

In another aspect, the present invention broadly resides in a nappy including
a pouch attached to the nappy; and
a change mat located in the pouch.

The features described with respect to one aspect also apply where applicable to all other aspects of the invention. Furthermore, different combinations of described features are herein described and claimed even when not expressly stated.

### BRIEF DESCRIPTION OF THE DRAWINGS

In order that the present invention can be more readily understood reference will now be made to the accompanying drawings which illustrate a preferred embodiment of the invention and wherein:

FIG. 1 is a schematic view of a nappy and a nappy accessory according to an embodiment of the present invention;

FIG. 2 is a schematic view of the nappy accessory of FIG. 1 attached to the nappy of FIG. 1;

FIG. 3 is a schematic view of the contents of the nappy accessory of FIG. 1;

FIG. 4 is a schematic view of a change mat according to an embodiment of the present invention; and

FIG. 5 is a schematic view of the change mat of FIG. 4 converted to a bag.

### DETAILED DESCRIPTION OF THE PREFERRED EMBODIMENT

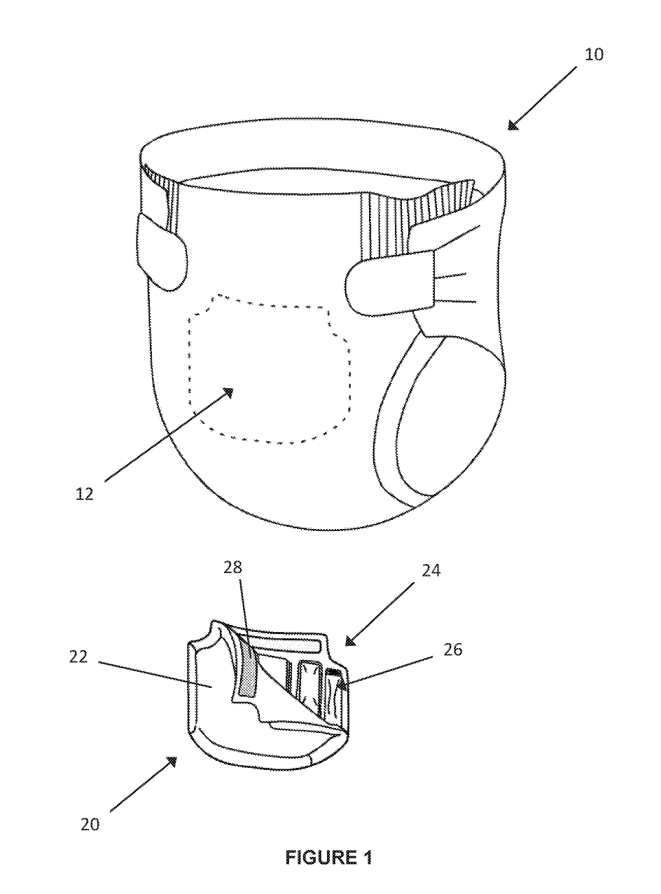

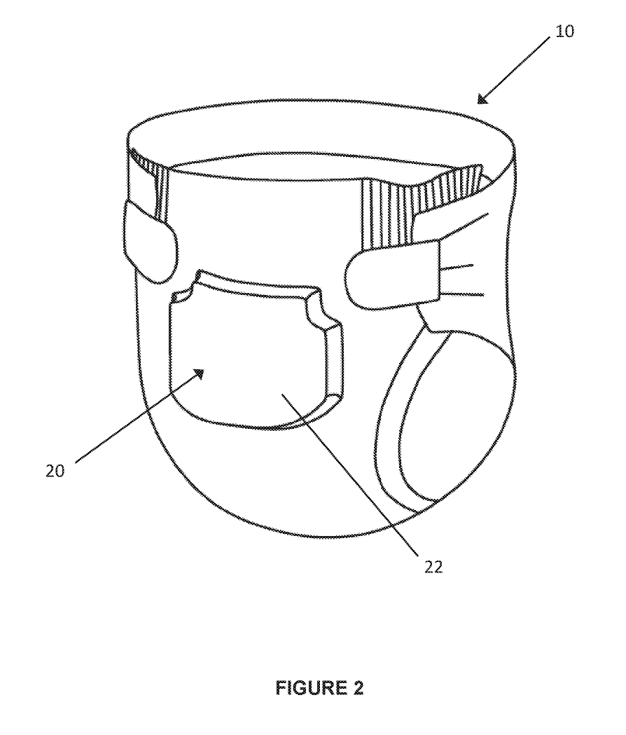

With reference to FIGS. 1 and 2, there is shown a nappy 10 and a nappy accessory 20 according to an embodiment of the present invention. FIG. 1 shows the location 12 where the nappy accessory 20 is attached to the nappy 10.

The nappy accessory 20 has a pouch 22. The pouch has an adhesive portion on a rear side thereof (not shown) which enables the pouch 22 to be attached to the nappy 10.

The pouch 22 has an opening 24 so that contents 26 of the pouch 22 can be accessed. The pouch 22 has an adhesive strip so that the opening 24 can be resealed.

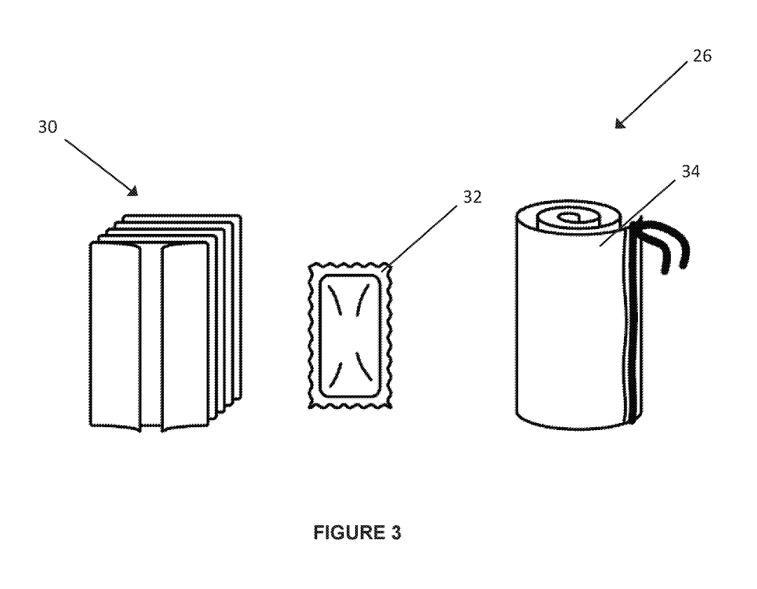

With reference to FIG. 3, the contents 26 of the pouch 22 in FIG. 1 are shown in more detail. The nappy accessory 20 of FIG. 1 includes a plurality of wipes 30. The nappy accessory 20 further includes a sachet of barrier cream 32. The nappy accessory 20 also includes a change mat 34. The change mat 34 is located in the pouch 22 (as seen in FIG. 1) in a folded and rolled condition.

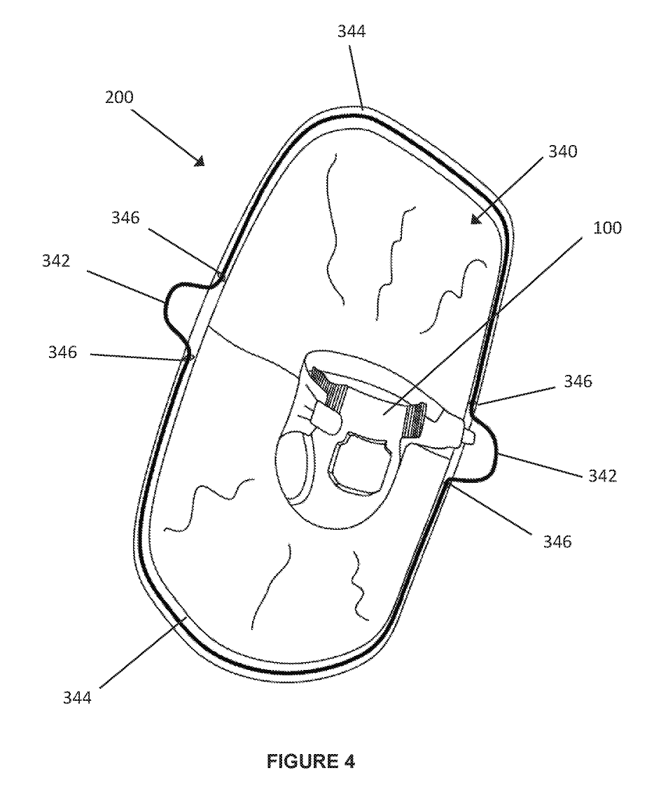

FIG. 4 shows a nappy accessory 200 including a change mat 340. FIG. 4 shows the change mat 340 in use with a soiled nappy 100 located on a top surface of the change mat 340. The change mat 340 is made from a thin plastic material so that it is light weight and easy to fold and/or roll up to store prior to use.

The change mat 340 includes a drawstring 342 located around a periphery of the change mat 340. A portion of the change mat 344 is folded over to retain the drawstring 342. The folded portion 344 includes apertures 346 so that the drawstring 342 can be accessed.

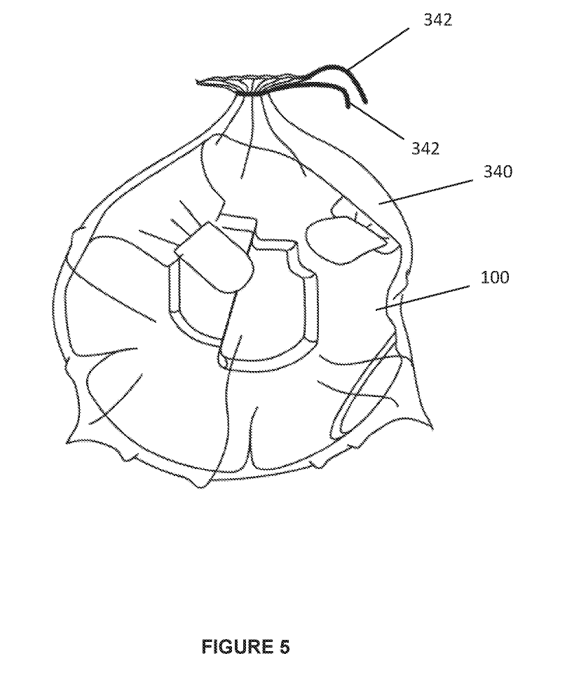

FIG. 5 shows the change mat 340 when the draw string 342 has been pulled. The change mat 340 has now converted to a bag containing the soiled nappy 100. Two opposed loops of the drawstring 342 can be tied together to securely close the change mat 340 so that the soiled nappy 100 is safely contained until the change mat 340 and the soiled nappy 100 can be properly disposed of.

### ADVANTAGES

An advantage of a preferred embodiment of the nappy accessory includes having a resealable pouch to allow access to the contents of the pouch when the nappy is not being changed. Another advantage of a preferred embodiment of the nappy accessory includes having the pouch resealable and the pouch releasably attached to a nappy so that the pouch can be reused. A further advantage of the preferred embodiment of the nappy accessory includes a convenient way to carry accessories needed to change a soiled nappy.

### VARIATIONS

While the foregoing has been given by way of illustrative example of this invention, all such and other modifications and variations thereto as would be apparent to persons skilled in the art are deemed to fall within the broad scope and ambit of this invention as is herein set forth.

Throughout the description and claims of this specification the word “comprise” and variations of that word such as “comprises” and “comprising”, are not intended to exclude other additives, components, integers or steps.

1. A nappy accessory including
a pouch attached or attachable to a nappy; and
a change mat located in the pouch.

2. A nappy accessory as claimed in claim 1 further including one or more of: one or more wipes located in the pouch, one or more wet wipes located in the pouch, and one or more packets of barrier cream and/or baby powder located in the pouch.

3. A nappy accessory as claimed in claim 1, wherein the change mat includes a drawstring located adjacent a periphery of the change mat, and wherein in use the drawstring can be pulled to convert the change mat into a bag.

4. (canceled)

5. A nappy including a nappy accessory as claimed in claims 1.

6. A nappy accessory as claimed in claim 1, wherein the pouch includes one or more internal pockets.

7. A nappy accessory as claimed in claim 1, wherein the change mat is releasably attached to an inside portion of the pouch.

8. A nappy accessory as claimed in claim 1, wherein the change mat includes a tab attached to an inside portion of the pouch.

9. A nappy accessory as claimed in claim 8, wherein the tab is tearable to detach the change mat from the pouch.

10. A nappy accessory as claimed in claim 3, wherein a portion of the change mat is folded over to contain the drawstring.

11. A nappy accessory as claimed in claim 10, wherein the folded over portion includes one or more apertures to access the drawstring.

12. A nappy accessory as claimed in claim 11, wherein the folded over portion includes two apertures to access the drawstring.

13. A nappy accessory as claimed in claim 12, wherein the drawstring can be pulled from both of the two apertures to convert the change mat into a bag and form two loops of drawstring, and the two loops of drawstring extending from the two apertures can be tied together to securely close the bag.

14. A nappy accessory as claimed in claim 1, wherein the pouch is resealable.

15. A nappy accessory as claimed in claim 14, wherein the pouch is resealable using an adhesive strip.

16. A nappy accessory as claimed in claim 14, wherein the pouch is resealable using a hook and loop fastener.

17. A nappy accessory as claimed in claim 1, wherein the pouch has a weakened portion which enables a user to open the pouch.

18. A nappy accessory as claimed in claim 17, wherein the weakened portion is a perforated portion.

19. A nappy accessory as claimed in claim 1, wherein the pouch is glued or heat welded to a nappy.

20. A nappy accessory as claimed in claim 1, wherein the pouch includes an adhesive portion having a removable backing to expose the adhesive portion, wherein the pouch is attachable to a nappy by adhering the adhesive portion to a portion of the nappy.

21. A nappy accessory as claimed in claim 1, wherein the pouch includes a hook fastener portion to releasably attach to fibres of an external fabric of a nappy.
